(19) United States
(12) Patent Application Publication
(10) Pub. No.: US 2009/0326069 A1
(43) Pub. Date: Dec. 31, 2009

(54) Azelaic acid ester compositions and methods for diagnosing and treating tissue conditions using azelaic acid ester compositions and proteinaceous biomarkers

(75) Inventors: Robert T. Streeper, Marion, TX (US); Elzbieta Izbicka, Marion, TX (US)

Correspondence Address: LAW OFFICES OF CHRISTOPHER L. MAKAY, 1634 MILAM BUILDING, 115 EAST TRAVIS STREET, SAN ANTONIO, TX, 78205-1763, US

(73) Assignee: BLTN, LLC

(21) Appl. No.: 12/459,338

(22) Filed: Jun. 30, 2009

Related U.S. Application Data
(60) Provisional application No. 61/076,769, filed on Jun. 30, 2008.

(51) Int. Cl. A61K 31/225; C07C 69/48; A61P 29/00

(52) U.S. Cl. 514/547; 560/190

(57) ABSTRACT

Therapeutic and cosmetic treatments are performed through the application of azelaic acid ester compositions to patients suffering from or exhibiting tissue inflammation. The diagnosis, characterization and treatment of a patient suffering from or exhibiting tissue inflammation may be enhanced by using selected proteinaceous biomarkers obtained from analysis of said markers by means of multiplexed immunoassay and comparison to the biomarker levels of healthy tissue.

### CROSS-REFERENCE TO RELATED APPLICATIONS

This present application claims all available benefit, under 35 U.S.C. §119(e), of U.S. provisional patent application Ser. No. 61/076769 filed Jun. 30, 2008. By this reference, the full disclosure of U.S. provisional patent application Ser. No. 61/076769 is incorporated herein as though now set forth in its entirety.

### BACKGROUND OF THE INVENTION

1. Field of the Invention

The present invention relates to diagnosis of tissue conditions using proteinaceous biomarkers and the use of azelaic acid ester compositions for mammalian therapeutic and cosmetological treatment.

2. Description of the Related Art

Diseases such as psoriasis, diabetes, rheumatoid arthritis, scleroderma, lupus, Crohn's disease, ALS, MS, and others raise the question of the origin or initiation of the autoimmune response that is the fundamental self-destructive malfunction of the immune system and its various components. It is well documented that all of these diseases are mediated through autoreactive cell mediated immune responses. The current methods of palliating these diseases revolve around inhibiting the immune response by the administration of immunotoxic drugs such as cyclophosphamide, methotrexate or more recently by the administration of biological response modifiers such as those that inhibit TNF function. TNF (cachexin or cachectin and formerly known as tumor necrosis factor-alpha) is a cytokine involved in systemic inflammation and is a member of a group of cytokines that stimulate the acute phase reaction. All of these treatments are administered based on the premise that the immune system has become self-reactive and/or overactive with a resulting detriment to the patient suffering from the condition and that it is the immune system or a component of the immune system that is primarily defective. Paradoxical though is the observation that in physiological states where the immune system activity is diminished, such as in patients undergoing antineoplastic chemotherapy or radiotherapy, both of which diminish immune response, these autoimmune diseases are often induced, aggravated or become clinically apparent. This phenomenon is observed in diabetics who go on to develop psoriasis or rheumatoid arthritis.

Thus one must ask, if the immune system causes the autoimmune diseases, why does decreasing the activity of the immune system often provoke the development of many of these same autoimmune diseases?

Diseases such as inflammatory dermatoses constitute a significant health burden. A large population study in Germany (Schaefer, I. et al., Dermatology 217(2) 169-172, Jun. 5, 2008) found the following:
Results: 48,665 persons (52.8% male) with a mean age of 43.2 years were examined. The following frequencies of chronic and acute inflammatory skin diseases were observed: contact eczema 8.9%, acne 4.2%, seborrheic eczema 3.4%, rosacea 2.2%, psoriasis vulgaris 2.1%, atopic eczema 1.4% and lichen ruber 0.07%. In total, dermatological findings needing treatment were observed in 19% of the study cohort; 1.3% of the participants presented a suspicious finding requiring diagnostics for skin malignancies. Conclusions: There is a high frequency of chronic inflammatory skin diseases in the general working population and a high need for dermatological treatment (emphasis added).

Acne is suffered to a greater or lesser degree by the entire population. In the United States, 75% of the population rates acne as a significant medical concern at some point in their lives.

Conventional wisdom maintains that acne is a disorder of the pilosebaceous unit, hair follicles, wherein commensal bacteria, most notably Proprionobacterium acnes, cause the cells of the hair follicle to become irritated which causes increased sebum secretion. In addition disorders of keratinization of the keratinocytes of the hair follicle causes the keratinocytes to increase the rate at which they mature and are shed into the lumen of the follicle. These factors are thought to combine to create a plug of shed cells and sebum mixed with overgrowing P. acnes resulting in the physical obstruction of the hair follicle. This obstruction is consistent with the observed characteristics of acne lesions, comedone formation and inflammation.

Thus treatments for acne have been designed which are directed to correction or alleviation of these perceived causes of disease, such as ‘normalization’ of keratinization, with the aim of correcting the excessive proliferative activity of the keratinocytes. Many drugs are used to normalize keratinization. Retinoids are one class of compounds used in the treatment of acne. Oral and topical retinoids are known to exert therapeutic effects beneficial for the treatment of acne by regulating gene activation and expression with the effect of controlling keratinocyte maturation. The so called keratolytic drugs such as benzoyl peroxide, salicylic acid, azelaic acid, and various enzymes having proteolytic activities that soften and digest shed keratinocytes are also used for the treatment of acne. Stronger acids such as glycolic acid, lactic acid and others essentially burn away the outer layer of skin cells are used in an effort to resurface the skin. These keratolytic drugs are used in an effort to remove keratinized cells and effect opening of clogged pores and hair follicles. A second therapeutic avenue that is used in the treatment of acne is through the reduction of sebum production or removal of excess sebum which can be achieved by washing the skin with soaps, cleansers or solvents or by the application of drugs such as retinoids which also reduce sebum production. The third and possibly the most often used pharmacologic intervention in acne is the application of various drugs intended to reduce the numbers of bacteria present on the skin. Examples of this class of drugs include oral and topical antibiotics, benzoyl peroxide, hexachlorophene and so on. Each of these therapeutic approaches has advantages and limitations, but none have proven to be universally curative. Even the best of these treatments can claim only 70% efficacy. Many of these drugs also have very significant drawbacks, for instance the retinoids must not be used by women who are or may become pregnant due to the high risk of causing birth defects.

Azelaic acid is a naturally occurring straight chain, 9 carbon atom saturated dicarboxylic acid obtained by oxidation of oleic acid or by chemical, physical or biological oxidation of free and esterified fatty acids. Azelaic acid is a metabolite of longer chain fatty acids in human bodies. It is found also in small amounts in the urine of normal individuals (Mortensen 1984), and in whole grain cereals and some animal products. In vitro, azelaic acid has been shown to be a competitive inhibitor of a number of oxidoreduction enzymes such as tyrosinase (Nazzaro-Porro et al, 1979), thioredoxin reductase (Schallreuter 1987), DNA polymerase (Galhaup 1989), and also of mitochondrial oxidoreductases in the respiratory chain (Passi et al 1984). In addition, azelaic acid is a potent inhibitor of 5-α-reductase (Stamatidas et al 1988). Azelaic acid is a scavenger of toxic oxygen species and also inhibits oxyradical activity in cell cultures (Passi et al 1991 & 1989).

Azelaic acid has been used clinically for many years in the treatment of acne vulgaris as well as in hyperpigmentary skin disorders (Fitton 1991). Azelaic acid has also has recently been studied for the treatment of papulopustular rosacea (Maddin 1999).

While azelaic acid has been used primarily in the treatment of dermatological conditions, because of some of its mechanisms of action, it has further clinical utility in conditions unrelated to the skin. Azelaic acid has been shown to have antiproliferative and cytotoxic action on the following tumor cell lines: human cutaneous malignant melanoma (Zaffaroni et al 1990), human choroidal melanoma (Breathnach et al 1989), human squamous cell carcinoma (Paetzold et al. 1989), and fibroblastic tumor cell lines (Geier et al 1986). Azelaic acid would presumably also have utility in the prevention and treatment of skin cancer as well as solar keratosis. Because of its mechanism of action as a potent inhibitor of 5-α-reductase, azelaic acid may be applicable to the treatment and prevention of benign enlargement of the prostate as well as cancer of the breast or prostate and other conditions in which 5-α-reductase is implicated in biological processes, such as hair loss.

Clinical applications of azelaic acid have included treatment of acne vulgaris, hyperpigmentary skin disorders, and papulopustular rosacea. Azelaic acid is marketed for the treatment of acne and rosacea in concentration not greater than 20% because of excessive skin irritation experienced by users. Azelaic acid is marketed for dermatologic indications in the product Skinoren (made by Schering) or Azelex (Allergan). Azelaic acid is prepared as a 20% concentration cream for topical application and it is commonly used in the treatment of inflammatory acne vulgaris. Azelaic acid at 15% concentration in a gel carrier is also marketed for the treatment of rosacea under the trade name Finacea. Higher concentrations of azelaic acid (>20%) have been found to be excessively irritating, limiting the maximum tolerated concentration to 20% or less.

U.S. Pat. Nos. 4,292,326; 4,386,104; and 4,818,768 describe azelaic acid as well as other dicarboxylic acids in the treatment of acne and melanocytic hyperpigmentary dermatoses. U.S. Pat. Nos. 4,713,394 and 4,885,282 describe azelaic acid as well as other dicarboxylic acids used in the treatment of non-acne inflammatory dermatoses and infectious cutaneous diseases such as rosacea, perioral dermatitis, eczema, seborrheic dermatitis, psoriasis, tinea cruris, flat warts, and alopecia areata. One of Thomfeldts' formulations comprises azelaic acid dissolved in a large proportion of ethanol. U.S. Pat. No. 6,451,773 describes a composition for treating acneiform eruption containing a chitosan having a molecular weight ranging from about 500,000 to about 5,000,000 g/mole and a degree of deacylation greater than 80% and an acid-form active ingredient such as azelaic acid for treating acne. U.S. Pat. No. 6,734,210 discloses the stable salts of azelaic acid with polycations.

Venkateswaran, U.S. Pat. No. 5,549,888, discloses a solution of active ingredients which includes azelaic acid and is partially solubilized by a glycol. It further teaches the use of glycol in combination with ethyl alcohol to solubilize the azelaic acid. Venkateswaran also teaches that the formulation has a pH between 2.5 and 4.0.

### SUMMARY OF THE INVENTION

In accordance with the present invention, compositions are produced and methods are disclosed for making decisions about the use of the compositions that rely on the employment of simple esters of azelaic acid for the treatment of conditions that involve, as part of their etiology intracellular and intercellular signaling mediated by the expression, synthesis, release and recognition of biological molecules that result in or precipitate inflammatory conditions that are not beneficial to the overall welfare of the host. The compositions of matter of the invention include esters of azelaic acid that modulate the expression, release, synthesis, recognition and action of biological molecules known to be integral signaling mediators involved in inflammatory processes important in human and other animal diseases. The compositions of matter of the invention further include mixtures of one or more azelaic acid esters with any or all of the pharmacological agents described herein. The azelaic acid esters are applied either alone or in various combinations with other pharmacologically active materials that benefit the patient by the amelioration, treatment or cure of a range of diseases mediated by intracellular and intercellular signal transduction molecules.

As used herein, the term “anti-inflammatory and/or chemo-protective” means the beneficial modulation, regulation or modification of intracellular and intercellular signaling in such a way as to benefit a mammal suffering from a disease having an inflammatory component. In addition, the term “‘inflammatory”’ includes all of those cellular and systemic responses of the host wherein there is a measurable change in the concentration of one or more intercellular or intracellular signaling molecules that involves the cells and tissues of the mammalian immune system. The term “treat” or “treatment” as used herein means a therapeutic intervention designed to cure a disease or palliate the symptoms associated with one or more diseases or conditions. The term “prevent” as used herein means that subsequent occurrences of symptoms and/or manifestations of disease are avoided or that the frequency between such occurrences is prolonged.

The azelaic acid esters of the invention have been found to exert distinct and unique anti-inflammatory and/or chemo-protective effects in human tissue which are different and distinct from the properties of azelaic acid itself. These esters of azelaic acid in addition have utility in increasing blood and other tissue or body fluid levels of the esters which facilitates the delivery of the esters for the treatment of a wide variety of conditions involving inflammation.

The present invention in addition is related to techniques, methods and reagents for a multifactorial assay for the facile diagnosis, characterization and treatment of inflammatory conditions of tissues, and, more particularly, the invention is related to a multimarker tissue diagnostic test for the diagnosis of disease and as an aid in the selection of treatments for the amelioration of diseases having an inflammatory component, including those of the skin and other body tissues. The invention is more specifically related to a method for guiding the selection of treatments and other therapeutic interventions so as to alter the body and tissue levels of proteins and peptides and other biological molecules that are significantly differentially expressed in the tissues of individuals suffering from inflammatory diseases, referred to herein as inflammatory biomarkers, and to how the application of certain treatments including azelaic acid esters produces an improvement in the disease status or symptoms of the patient secondary to alterations in the quantities of the inflammatory biomarkers present in the tissues of a patient suffering from a disease having an inflammatory component.

The present invention discloses lipid soluble esters of azelaic acid, methods for the selection thereof for the treatment of various diseases, methods for the use thereof and synthetic methods for their preparation.

The esters of the invention have significant therapeutic advantages over azelaic acid and its salts as described in the patent and scientific literature. The azelaic acid esters are not acidic and are therefore much less irritating. The various azelaic acid esters of the invention also produce distinctly different biomarker response patterns and physiological effects when applied to mammalian tissues. The various esters of azelaic acid also have different and complementary activities that lend to their use singly and as mixtures in the treatment of inflammatory diseases in various rationally selected combinations. The invention also encompasses quantitative measurements of tissue levels of inflammatory biomarkers to form a biomolecular basis for characterizing tissue responses to treatment with certain mixtures of azelaic acid esters. The invention further includes the utilization of the derived pattern of biomarkers as a guide for the selection of treatments and for the optimization of the composition of the mixtures of azelaic acid esters to be used. The azelaic acid esters can be used singly or in mixtures that may in addition be used in combination with other drugs that is used to promote and induce beneficial therapeutic effects in a patient suffering from inflammatory disease.

### DETAILED DESCRIPTION OF THE PREFERRED EMBODIMENTS

The present invention and its research show that immunomodulatory stimuli play a role in a range of human and animal diseases. Stimuli such as an underlying systemic infection or inhabitation by viruses, bacteria, dermatophytes, yeasts, mites, mycoplasmas, fungi or other parasitic organisms or chemical insults due to allergens or sensitizers elicit inflammatory responses by the cells of the organisms' immune system. The elaboration and release of immune-stimulatory and immunomodulatory biological molecules initiates a cascade of immune system activity directed at destruction of the invading pathogens or for the elimination of the damage due to chemical or other insult. It is also well known that such immune system activity may after stimulation begin to recognize and react to host tissues or cells resulting in the development of autoimmune diseases. In addition, the immune system may begin to react to various combinations or products of host and exogenous antigens. These neoantigens or super antigens may consist in part of haptens derived from the pathogen and in part haptens derived from or present on the host cells or tissues.

It is known that the immune system then elaborates an immune response to the antigens, be they exogenous or endogenous, and produces various inflammatory mediators that may directly or indirectly damage tissue. If the immune response is excessive or continues for too long a time, tissue damage can occur that precipitates the characteristic signs, symptoms and effects of the inflammatory or autoimmune disease. The immune system may become inappropriately self reactive due to stimulation by one or more exogenous stimuli alone or through the actions of multiple stimuli acting in concert. The stimulus may also act as an adjuvant wherein the immune system activation is amplified and then subsequently over reacts or inappropriately identifies self antigens as targets resulting in an anti-self immune response. In addition it is known that in some patients suffering from inflammatory diseases there is a state of immune hyper-reactivity that can be triggered or exacerbated by the presence of stimuli such as sunlight, heat, certain foods, emotional stressors etc. A well known example of diseases of immune hyper-reactivity is seasonal allergy or hay fever.

It can be concluded based both on evidence presented in the scientific and medical literature and the observations made in the studies associated with the present invention, that many human diseases including long term or chronic human diseases have a component that may be justifiably characterized as an autoimmune disorder or as an immune hyper-reactivity disorder. Such diseases include many diseases of the skin and other body tissues.

For example, it can be shown that the fundamental cause of the multiple diseases known clinically and colloquially as acne stem from an inappropriate and counterproductive immune system reactivity. In this state of hyper reactivity, immune effector cells primed for the recognition of bacterial antigens, become over-reactive to and therefore intolerant of the presence of bacterial antigens. The reasons for this over-reactive state are at present incompletely understood. This anti-self, auto-immune or immune hyper-reactivity leads to excessive immune cell and immune system activity mediated by the production and release of pro-inflammatory signaling molecules and the mobilization of various immune effector cells. These signals cause the recruitment of additional immune effector cells to the site of initial stimulation and produce broader tissue inflammation. This inflamed tissue is then vulnerable to bacterial efflorescence, which further facilitates or drives forward the inflammatory cycle. Thus, by decreasing inflammatory signal production and release, the azelaic acid esters of the invention dampen the inflammatory cycle and consequently decrease tissue inflammation.

The inflammatory response under normal circumstances is an essential element of the host immune response. However when the immune response becomes disproportionate, disregulated or becomes auto-reactive it is necessary to control or down-regulate the immune response to prevent damage to tissues. Controlling inappropriate inflammation is central to the treatment and resolution of many diseases, ranging from microbial infections to auto-immune diseases. In many of these diseases the inflammatory response is initiated and then goes on to become disregulated.

Recently a variety of drugs that aim to control destructive inflammatory response by specifically targeting and disabling certain key inflammatory mediators have come into clinical use. These drugs are known as a class as biological response modifiers. Biological response modifiers, such as the anti-TNF agents, control the immune response by inactivating certain key inflammatory biomarkers. These agents, while effective, have proven to have significant drawbacks. For instance, certain anti-TNF drugs have been shown to cause lymphoma in some patients. Moreover, at least one anti-TNF drug has been removed from the market because of the development of progressive multifocal leukoencephalopathy in some patients. The specificity and potency of these drugs is the fundamental cause of these side effects.

The azelaic acid esters of the invention on the other hand act to decrease immune system inflammatory activity in such a way as to modulate the release of inflammatory mediators and decrease the level of activation of immune system cells in a way as to be beneficial to the host. The azelaic acid esters exert these effects in a broad and reversible fashion that does not result in the long term or profound down-regulation of any particular inflammatory mediators or pathways. The utility of the azelaic acid esters in controlling inflammatory diseases can be illustrated by making reference to acne as discussed herein. Particularly, controlling the tissue inflammation present in acne can greatly palliate the disease, and by the application of selected mixtures of azelaic acid esters it is possible induce significant improvement in disease status.

A discussion of all of these immune system mediated inflammatory disorders would be too lengthy to describe herein, therefore, acne is used as an example since it is well known and is not commonly thought of as an immune system disorder or disease.

While it is rarely dangerous, acne causes significant psychological distress in sufferers. And there are enormous gaps in the understanding of the etiology of the disease, with consequent inappropriate attempts at therapeutic intervention.

Acne has been treated with numerous pharmacological agents with varying degrees of success. There is as yet no agent that offers safe and universally effective treatment for acne.

The present invention describes a treatment for inflammatory conditions that addresses these and other needs. Azelate esters are non-toxic, non-irritating agents acting directly on the cells and tissues to alleviate inflammation. Azelaic acid esters are relatively easy to produce and the process is commercially scalable. Azelaic acid esters are in addition relatively inexpensive to produce. Azelaic acid esters are unique in the speed of action and the degree of palliation of the inflammation and for treating diseases having an inflammatory component.

The present invention discloses the use of esters of azelaic acid, compositions including mixtures of azelaic acid esters having complementary anti-inflammatory and/or chemo-protective activities, compositions including mixtures of azelaic acid esters with other drugs, methods for selecting appropriate mixtures thereof and synthetic methods for their preparation. The esters of azelaic acid of the invention have utility in increasing blood and other tissue or fluid levels of active drugs, as well as treating or preventing a wide variety of conditions related to inflammation of the body tissues.

Thus, in one embodiment, a mixture of azelaic acid esters is administered to a warm-blooded animal in need thereof. The diseases amenable to treatment with azelaic acid esters include all diseases in which inflammation is part of the disease process. Such conditions include but are not limited to: Inflammatory conditions of the circulatory system such as atherosclerosis and inflammatory vasculopathies, inflammation of the heart and valves of the heart. Inflammatory conditions of the lymphatic system such as lymphadenitis. Inflammatory conditions of the body tissues due to or secondary to microbiological infection, thermal, or mechanical trauma. Inflammation-induced bone remodeling in periodontal disease and other bone diseases, inflammatory conditions of the gastrointestinal tract such as Crohn's disease, inflammatory bowel disease, Bechets disease, abscesses and inflammations of the oral cavity due to trauma and/or infection. Inflammatory conditions of the ears and auditory canals and associated structures such as the inflammation secondary to otitis media. Inflammatory conditions of the joints or connective tissues such as bursitis, arthritis and arthralgia. Inflammatory conditions due to allergic stimuli such as atopic dermatitis, contact dermatitis and chemical sensitivity. Inflammatory conditions due to or secondary to the bites or stings of insects. Inflammatory conditions wherein the cellular and or mitochondrial levels of adenosine triphosphate become deranged in such a way as to precipitate disease or the symptoms thereof.

In yet a further embodiment, the mixtures of azelaic acid esters are administered to a warm blooded animal to prevent and or treat the following conditions: Aging of the skin, cancer, HIV, alopecia, solar keratosis, benign prostatic hypertrophy, prostate cancer, acne, bacterial infection, malignant melanoma, hair loss, bladder cancer, rosacea, conditions in which tyrosinase activity needs to be modulated, melasma, conditions in which 5-α-reductase activity needs to be modulated, conditions related to excessive expression of reactive oxygen species such as stroke, heart attack and other pathological ischemias, lentigo maligna, hyperpigmentation associated with burns and other physical trauma, viral infections, and herpes labialis and genitalis, and viral hepatitis. Still other conditions could include, but are not limited to:

inflammatory disease and/or allergic reaction;

autoimmune disease or reaction;

infection by or allergic reaction to viral infection;

infection by or allergic reaction to bacterial infection;

infection by or allergic reaction to fungal infection;

infection by or allergic reaction to mycoplasma infection;

infection by or allergic reaction to bacillary infection;

infection by or allergic reaction to mycobacterial infection;

infection by or allergic reaction to one or more Leishmania species;

infection by or allergic reaction to one or more strains of yeast;

infection by or allergic reaction to one or more amoebic parasites;

infection by or allergic reaction to one or more worms including cestodes, nematodes and trematodes;

infection by or allergic reaction to one or more filarial parasites;

infection by or allergic reaction to one or more Staphylococcus species;

infection by or allergic reaction to one or more Camphylobacter species;

infection by or allergic reaction to one or more Proprionobacterium species;

infection by or allergic reaction to one or more Mallasezia species;

infection by or allergic reaction to one or more human papilloma virus species;

infection by or allergic reaction to one or more herpes simplex virus species;

infection by or allergic reaction to one or more Demodex species;

infection and or allergic reaction by one or more Wollbachia species;

infection and or allergic reaction by one or more Leishmania species;

infection and or allergic reaction by one or more Trachomatidis species;

infection and or allergic reaction by one or more Trycophyton species;

infection and or allergic reaction by one or more Mycobacterium species;

infection and or allergic reaction by one or more Microsporum species;

infection and or allergic reaction by one or more Epidermophyton species;

infection and or allergic reaction by one or more Candida species;

infection and or allergic reaction by one or more Aspergillus species;

neoplasia or cancer such as inflammatory breast cancer;

arthritis;

bursitis;

inflammation of the joints or other skeletal structures including osteochondrodysplasia, osteomalacia, Paget's disease, multiple myeloma or osteoporosis;

chondritis;

psychological depression or other affective disorder having an inflammatory component;

cardiovascular disease;

high blood pressure;

alcohol related illnesses;

systemic inflammatory syndrome;

septic shock or septicemia;

as a chemoprotectant against the deleterious effects of chemical and biological agents;

preventing chemical or biological damage and subsequent suffering resulting from the action of one or more biological or chemical irritants, toxins, poisons, metabolites or molecules arising from any cause with the aim of preventing or minimizing said damage or palliating the symptoms secondary to the damage;

Other aspects of the present invention will become evident upon reference to the detailed description.

In a preferred embodiment, a treatment and a method of treating a disease having an inflammatory component includes a pharmaceutical composition comprising at least one azelaic acid ester in a pharmaceutically acceptable vehicle or diluent. It is understood that the composition may further comprise a second or more highly lipophilic azelaic acid derivatives. This composition may preferentially include one or more or two or more esters of azelaic acid having a combined concentration of between 1 and 100 percent by weight. A preferable concentration of azelaic acid esters in such a composition for application to the skin may have a total concentration of azelaic acid esters of between 5 and 25 percent by weight. More preferably, the combined concentration of azelaic acid esters for the treatment of skin diseases is between 10 and 20 percent by weight. The relative proportion of the various azelaic acid esters present in the mixed esters is determined by reference to the biomarker modulation pattern of the individual esters as they relate to the particular inflammatory mediators that characterize the inflammatory disease being treated. For instance, a mixture of azelaic acid esters constituted of approximately 5 weight percent dimethylazelate, 90 weight percent diethylazelate and 5 weight percent diisopropylazelate has been found to be particularly suited to the treatment of acne and other inflammatory diseases of the skin. These various mixtures of azelaic acid esters, tailored to the treatment of particular conditions by reference to the biomarker modulation patterns of the invention, can be combined with one or more additional fatty diacid derivatives and/or one or more other pharmacologically-active compounds, and particularly one or more anti-inflammatory and/or chemo-protective, anti-tumor, anti-fungal, anti-viral or anti-bacterial compounds. Compounds could include, but are not limited to:

salicylic acid or salicylic acid salts or derivatives;

benzoic acid or salts of benzoic acid;

benzyl benzoate;

resorcinol;

eucalyptus oil;

4-ethoxy benzaldehyde;

retinoic acid, retinol or other synthetic or naturally occurring retinoid;

a tetracycline antibiotic;

doxycycline and/or doxycycline hyclate;

clindamycin and/or clindamycin phosphate;

alpha difluoromethylornithine;

benzoyl peroxide;

adapalene;

tretinoin;

isotretinoin;

vitamin D;

nicotinic acid its salts and esters;

an antihistamine;

diphenhydramine;

physiologically active steroid;

hydrocortisone;

a vanilloid receptor agonist;

capsaicin or its congeners;

azelaic acid and its salts;

extracts of plants having complementary additive or synergistic effects when used in combination with one or more azelaic acid esters;

extracts of Camillus sinensis;

extracts of Nerium oleander;

extracts of Aloe Barbadensis;

an inhibitor of cyclooxygenase enzymes;

indomethacin;

acetylsalicylic acid;

diclofenac;

esters of salicylic acid;

nicotinamide or nicotinic acid;

vitamin E its salts derivatives and compounds;

chromium picolinate;

zinc metal and its salts;

undecylenic acid its esters and salts;

zinc undecylenate;

anti-fungal compounds or drugs;

ketoconazole;

miconazole;

voriconazole;

itraconazole;

anti-viral compounds;

anti-mycobacterial compounds;

anti-neoplastic compounds;

anhelminthic compounds;

insecticidal compounds;

compounds having insect repellant properties;

nematocides;

therapeutic antimonial compounds;

anti-malarial compounds;

compounds that enhance transdermal drug delivery such as dimethylsulfoxide;

antimicrobial peptides or proteins or their derivatives;

lipids or lipid esters;

ceramides;

kojic acid and/or its salts and derivatives;

psoralen;

methotrexate;

5-fluorouracil;

anti-viral compounds;

a TNF modulator or blocker; and

a NFκB modulator or blocker.

The methods of the invention may thus entail the administration of one, two, three, or more azelaic acid esters in conjunction with other pharmacologically active molecules. The maximum number of types of molecules that may be administered is limited only by practical considerations, such as the particular effects of each compound.

The invention further includes the use of the mixed azelaic acid esters according to Formula I in the manufacture of a medicament for oral, topical, intravenous, transdermal, rectal, vaginal, intraperitoneal, trans-placental, ocular, nasal or parenteral delivery with the intention of relieving disease conditions in a mammal.

The present invention provides a method for diagnosis of the presence of inflammatory disease in a patient, comprised of measuring tissue levels of a panel of markers of inflammation in which significant differences in the tissue concentrations of the markers in the tissue is compared to healthy matched control tissue that correlates or is associated with the presence of inflammatory diseases, the disease causing organism or defect or the risk of developing inflammatory diseases.

In spite of considerable research into therapies for inflammatory diseases and inflammatory dermatoses, the etiology of the diseases is often difficult to divine and they can be therefore difficult to treat effectively. Accordingly, there is a need in the art for improved methods for detecting and treating such disorders, and designing pharmacological and therapeutic interventions to mitigate and or cure these types of diseases. The present invention fulfills these needs and further provides other related advantages.

The present invention fulfills this need by providing methods of treatment of inflammatory dermatoses in a patient by determining tissue levels of biomarkers, such as for instance by using Luminex xMAP® (Luminex Corp., Austin, Tex.) technology, which allows for simultaneous measurement of the biomarkers in tissue. The panel of the tissue markers offers extremely high predictive power for discrimination of inflammatory diseases from both healthy control patients and from patients with inflammatory diseases or for patients at risk of developing inflammatory diseases. The methods of the invention allow for rapid, early diagnosis of inflammatory diseases including inflammatory dermatoses with extremely high sensitivity and with the necessary degree of specificity to be clinically useful in disease diagnosis and additionally for the selection of appropriate therapeutic interventions.

In particular, the present invention provides a method for diagnosis of the presence of inflammatory diseases such as inflammatory dermatoses in a patient comprising determining tissue levels of markers in a tissue marker panel, using those levels and patterns of inflammatory biomarkers to inform the selection of therapeutic interventions designed to modulate the levels of inflammatory biomarkers. The invention further makes use of inflammatory biomarker levels as reliable indicators of the efficacy of the interventions and for the monitoring of the response of the inflammatory diseases to the application of the interventions.

The present invention also provides a method for diagnosis and characterization of the presence of inflammatory diseases or susceptibility to the development of inflammatory diseases in a patient, comprised of measuring tissue levels of a panel of markers in which significant differences in the tissue concentrations of the markers in the diseased tissue is compared to healthy matched controls and how the differences correlate to or are associated with the presence of inflammatory diseases or the development of inflammatory diseases or the presence of disease causing organisms or a defect in cellular or organismal physiology that induces disease or the presence of one or more is risk factors for the development inflammatory diseases.

The invention further provides a method for the identification of patterns of altered protein expression levels in the tissues of a patient indicative of pathological disorders of tissue. The method includes: Obtaining one or more samples of tissue or bodily fluid from the patient; subjecting the samples to multiplexed immunoassay using automated bead based technologies to capture tissue proteins; analysis of the captured and bead-bound proteins using multi-channel automated identification and quantification instrumentation followed by statistical analyses and classification of the derived data using various computational pattern matching techniques of tissue quantities of captured proteins derived from said analyses. Data thus derived are used to classify pathologies and interpret and measure responses to treatments based on pattern matching of patient protein expression profiles against reference ranges characteristic of the particular pathologies. Thus the invention affords a technique for the detection and diagnosis of tissue pathologies and for the measurement and characterization of responses to treatments and pharmacological interventions. The invention also comprises the characteristic reference ranges for each identified biomarker taken in isolation and in combination with other markers revealed herein as they are indicative of tissue pathology. These data may then be used to design a pharmacological or other therapeutic intervention consisting of the application of mixtures of one or more pharmacologically active agents including as part of the treatment one or more esters of azelaic acid.

The invention is in addition related to directed application of methods and reagents for a multifactorial assay for the rapid characterization and design of therapeutic interventions and treatment of inflammatory diseases including inflammatory dermatoses. In this aspect, the invention is related to a multimarker tissue diagnostic test for the diagnosis and treatment of inflammatory diseases, especially of the skin and other tissues. The invention is more specifically related to proteins, peptides and signaling molecules that are significantly differentially expressed in the tissue of individuals with inflammatory diseases. Such quantitative measures of proteins are useful for the diagnosis of inflammatory diseases and for selection of treatments for the palliation and/or cure of inflammatory diseases.

It will be appreciated by those skilled in the art that changes could be made to the embodiments described herein without departing from the broad inventive concept thereof. It is understood, therefore, that this invention is not limited to the particular embodiments disclosed, but it is intended to cover modifications that are within the spirit and scope of the invention, as defined by the appended claims.

The mixtures of azelaic acid ester derivatives of the present invention are certain esters that show efficacy in the treatment of inflammatory diseases and which are suited to incorporation in pharmaceutical formulations.

The compounds illustrated in Formula I and used in the present invention are esters of azelaic acid suitable for treatment of inflammatory diseases.

R₂OOC—(CH₂)n-COOR₁ Formula I:

Examples of suitable straight-chain alkyl groups (R1 and R2) in Formula I include methyl, ethyl, propyl, butyl, pentyl, hexyl, heptyl, octyl, dodecyl, palmityl, stearyl and the like groups.

Examples of suitable branched chain alkyl groups include isopropyl, sec-butyl, t-butyl, 2-methylbutyl, 2-pentyl, 3-pentyl and the like groups.

Examples of suitable cyclic alkyl groups include cyclopropyl, cyclobutyl, cyclopentyl and cyclohexyl groups.

Examples of suitable “alkenyl” groups include vinyl (ethenyl), 1-propenyl, i-butenyl, pentenyl, hexenyl, n-decenyl and c-pentenyl and the like.

The groups may be substituted, generally with 1 or 2 substituents, wherein the substituents are independently selected from halo, hydroxy, alkoxy, amino, mono- and dialkylamino, nitro, carboxyl, alkoxycarbonyl, and cyano groups.

By the expression “phenalkyl groups wherein the alkyl moiety contains 1 to 3 or more carbon atoms” is meant benzyl, phenethyl and phenylpropyl groups wherein the phenyl moiety may be substituted. When substituted, the phenyl moiety of the phenalkyl group may contain independently from 1 to 3 or more alkyl, hydroxy, alkoxy, halo, amino, mono- and dialkylamino, nitro, carboxyl, alkoxycarbonyl and cyano groups.

Examples of suitable “heteroaryl” are pyridinyl, thienyl or imidazolyl.

As noted herein, the expression “halo” is meant in the conventional sense to include F, Cl, Br, and I.

Also included are all molecules of the aforementioned types including substitutions of 1 or more deuterium atoms in the place of one or more hydrogen atoms. Such substituted molecules are well known in the art to posses different pharmacological and pharmacodynamic properties relative to those of the un-substituted molecules that will give rise to therapeutic advantages such as longer half life, altered receptor affinity and other such effects encompassed within the realm of metabolic differences due to heavy isotope effects.

Among the compounds represented by the general Formula I, preferred compounds are such in which R1 and R2 are the same and is one of the following groups:

Methyl, ethyl, n-propyl, iso-propyl, n-butyl, iso-butyl, sec-butyl, t-butyl, n-pentyl, 2-pentyl, 3, pentyl, sec-pentyl, iso-pentyl, neo-pentyl, n-hexyl, 2-hexyl, 3-hexyl, sec-hexyl, iso-hexyl, cyclo-hexyl, palmityl, stearyl, methoxyethyl, ethoxyethyl, benzyl and or nicotinyl.

Among the compounds represented by the general Formula I, preferred compounds are such in which R1 and R2 are different and is one of the following groups:

Methyl, ethyl, n-propyl, iso-propyl, n-butyl, iso-butyl, sec-butyl, t-butyl, n-pentyl, 2-pentyl, 3, pentyl, sec-pentyl, iso-pentyl, neo-pentyl, n-hexyl, 2-hexyl, 3-hexyl, sec-hexyl, iso-hexyl, cyclo-hexyl, palmityl, stearyl, methoxyethyl, ethoxyethyl, benzyl and or nicotinyl.

And the other, R2, is also taken from the above list but is not the same as R1.

Other preferred compounds are such in which R1 is hydrogen and R2 is one of the groups listed above, or R2 is hydrogen and R1 is one of these groups.

The compounds of Formula I are esters (mono and di-esters) of azelaic acid formed either at C1 or C9, or at both carboxyl groups. As will be described herein it has now surprisingly been found that compounds of Formula I, in contrast to azelaic acid itself, are highly useful in the treatment of inflammatory diseases and conditions.

The compounds of Formula I can be prepared by various methods as already described in the literature for a number of azelaic acid esters (see the references cited above). A large number of methods are known to the art that will allow a skilled practitioner to produce the claimed composition of matter or its analogs and homologs. Among these are for instance: The direct formation of the ester from the requisite acid and an alcohol. This condensation may be achieved by the dehydration of the reaction mixture with a suitable agent or by heating a mixture of the acid and alcohol. Commonly used dehydrating agents and methods include, heat, concentrated acids such as sulfuric acid, acid anhydrides such as phosphorous pentoxide, gaseous acids such as hydrogen chloride gas introduced into a solution of the acid in the requisite alcohol, solution chemistries formed by reaction mixtures such as iodine or bromine with sodium hypophosphite or red phosphorous that generate hydriodic acid in-situ which then goes on to promote the formation of the ester by dehydration or transient organohalide formation, and so on. This listing should not be taken as being all-inclusive or exhaustive for there are many additional dehydration mediated esterification methods are known to the art.

A second major set of synthetic strategies comprise the methods wherein an activated intermediate of either the acid or the alcohol is formed which is then further reacted with the appropriate esterifying acid or alcohol to produce the desired ester. Among these are reactions of an alcohol with an activated form of the acid. Activated forms of the acid include acid halides, acid anhydrides including both homo and hetero anhydrides, the reaction of the internal anhydride of the parent acid with the requisite alcohol, esters and anhydrides of both the acid and the alcohol which are formed by reaction of the requisite acid or alcohol with p-toluene sulfonyl chloride to produce the tosyl anhydride or ester which is subsequently reacted with the alcohol or acid respectively to produce the desired final ester. Similarly one could substitute a simple organic acid anhydride, such as acetic acid anhydride, for the p-toluene sulfonyl chloride. In addition one could start with one ester selected from among the desired compositions of matter and by the means of solution of the ester in a desired alcohol in the presence of an appropriate acidic or basic catalyst effect a conversion of the starting ester of the acid to an ester wherein the alcohol becomes that in which the reaction is carried out which method is also known to the art as trans-esterification.

For example, one could start with the dimethyl ester of the acid and by solution of the ester in ethanol in the presence of an acid or base one could cause the facile formation of the diethyl ester of the acid. In addition, if a mixed ester of the acid were desired, one could utilize an appropriately composed solution of the two or more desired alcohols in any of the methods herein described.

One could resort to the use of halogenated intermediates or ingredients to form the required esters. For example, thionyl chloride will chlorinate both acids and alcohols, thereby resulting in the acyl and alkyl chlorides. These acyl and alkyl chlorides may then be further reacted with the desired alcohol or acid respectively to produce the desired ester products. Other common halogenating agents include for example oxalyl chloride and the chlorides and bromides of phosphorous such as phosphorous penta or trichloride and penta or tribromide or phosphorous oxychloride.

Finally, it is commonly practiced to form esters through the action of a strong base on a mixture of the acid and the alcohol. Examples of strong bases include lithium aluminum hydride and other metal hydrides, alkali metal alkoxides such as sodium ethoxide and diisobutyl aluminum hydride and so on.

This listing of materials and methods should not be interpreted to be limiting, exhaustive or all-inclusive but is merely presented for illustration of the claimed possible methods. In addition, any of the above methods may be used with appropriate modifications of the reactants and conditions to produce monoesters of the diacid, homo-diesters of the diacid or hetero-diesters of the diacid.

One method that has been utilized for efficient preparation of the homo-diester is through dissolution of azelaic acid in anhydrous alcohol. Through this solution anhydrous hydrogen chloride gas is passed with stirring at room temperature at a slow rate. After several hours of reaction the solution becomes saturated with hydrogen chloride and the gas can be turned off. Stirring at room temperature is continued for a time ranging from a few minutes to several hours as is necessary to ensure quantitative formation of the desired ester. To drive the reaction to completion and drive off the dissolved hydrogen chloride the solution is moderately warmed to effect gentle reflux of the solvent. The hydrogen chloride is vented in a safe manner. The solution of the ester in alcohol is then reduced in volume by heat or vacuum distillation. The crude ester is then washed several times with water containing a base such as sodium bicarbonate to remove both the remaining hydrogen chloride and any unreacted acidic reaction products or the starting acid. The washed ester is then separated from the wash solutions and washed several additional times with pure water. The solutions are allowed to separate and the water is discarded. The ester is then mixed with a suitable dessicant such as anhydrous magnesium sulfate to remove any remaining residual water. The ester is then further purified, as by fractional distillation at reduced pressure, and analyzed, as by gas chromatography mass spectrometry, to the degree necessary to produce an active pharmaceutical ingredient (API), that is suitable for the treatment of mammalian health disorders, including disorders of the skin.

As mentioned above, this invention is generally directed to lipophilic esters of azelaic acid. Such azelaic acid esters, when administered to a warm blooded animal in need thereof, have utility in the prevention or treatment of conditions enumerated herein in warm blooded animals, including humans.

It has been found that the esters of azelaic acid have good and beneficial characteristics that are such as to render them particularly suitable both for use in pharmaceutical formulations and in cosmetics. Owing to the simple conception and low cost of the present invention, the procedures described in this invention easily lend themselves to the adaptation of the preparation methods on an industrial scale.

The examples given herein illustrate the preparation of certain esters of azelaic acid. Only a few of the many possible embodiments that may be anticipated are shown by these examples which are intended to define, in a non-limiting sense, the scope encompassed by the invention.

Detailed descriptions of the experiments used in identification and preparation of the biological marker response profile of human skin in response to application of some azelaic acid esters are given in Example 1.

### EXAMPLE 1

A study that evaluated multiplexed immunoassays to identify panels of biomarkers for efficacy of novel therapeutics for dermatologic conditions in a preclinical ex vivo model.

Methods: A human EpiDerm™ model was used to compare modulation of skin irritation due to a topical exposure to 1.5% croton oil (CO) as a single agent or concurrent with either 0.5% indomethacin or 25% DIETHYLAZELATE. Following 18-hour exposure, toxicity was assessed by MTT conversion. Levels of 40 human chemokines, cytokines, and PGE(2) in conditioned media and tissue extracts were measured using multiplexed Luminex xMAP® immunoassays. Inter-group differences were assessed by Student t-test.

Results: Toxicity of DIETHYLAZELATE was comparable with that of indomethacin by MTT. Over 50% (21/40) of the examined analytes showed highly significant inter-group differences (p<0.0002). In comparison with CO, DIETHYLAZELATE alone downregulatecd 14 and upregulated 1 medium marker; 9 tissue markers were downregulated. Respective numbers for indomethacin alone were: medium (11 down) and tissue (3 down). Concurrent DIETHYLAZELATE and CO downregulated 10 and upregulated 3 medium markers; respective numbers for indomethacin were 12 down and 4 up. Interestingly, DIETHYLAZELATE plus CO downregulated 12 tissue markers versus 3 down and 5 up for indomethacin in the same regimen.

Conclusions: The results demonstrate superior anti-inflammatory and/or chemo-protective activity of DIETHYLAZELATE versus indomethacin and support an extended multiplexed immunoassay-based analysis of skin biomarker profiles in the EpiDerm™ model as robust tools in development of novel agents for treatment of skin diseases and for analysis of irritancy of cosmetics and other agents.

It can be shown that based on known biochemical modulations taking place in the above diseases that any of the following could be significantly modulated by the application of an irritant or counter irritant molecule or mixture of molecules. Such markers being: Adenosine triphosphate (ATP) Adiponectin, PAI-I (active/total), Resistin, NGF, MPO, sE-Selectin, sICAM-I, sVCAM1,CRP, SAA, SAP, Fibrinogen, Haptoglobin, NT-proBNP, EGF, Eotaxin, Fractalkaline, G-CSF, GM-CSF, IFN-y, IL-10, IL-12 (p40), IL-12 (p70), IL-13, IL-15. IL-17, IL-1a, IL-1α/β, IL-2, IL-4, IL-5, IL-6, IL-7, IL-8, IL-Ira, IP-10, MCP-I, MIP-1α, MIP-1β, TGFα, TNFα, VEGF, sCD40L, RANTES, Amylin (Total), C-Peptide, GLP-1/Amylin, Glucagon, Insulin, Leptin, MIF, sFas, sFasL, sVCAM-I, sICAM-1, AI, All, B, CII, CIII, E, Fibronectin, Cortisol, Keratin-6, Keratin-I, Keratin-10, Keratin-II, Involucrin, TGFB1, FSH, LH, TSH, Prolactin, GH, ACTH, CRP, ENA-78, Eotaxin, FGF-basic, G-CSF, GM-CSF, GRO-α, Leptin, MCP-3, MIG, NGF, PDGF-BB, TGF-pl, TNFβ, CTACK, FGF-basic, HGF, ICAM-I, IFNα, IL-2ra, IL-16, IL-18, LIF, M-CSF, MIF, MIG, β-NGF, SCF, SCGF-β, SDF-1α, EGF, HGF, I-TAC, MMP-1, MMP-2, MMP-3, MMP-7, MMP-8, MMP-9, MMP-12, MMP-13, Angiopoietin-2, Follistatin, PECAM-I, C-Peptide, Ghrelin, GIP, OLP-1, Glucagon, Insulin, PAl-I, Visfatin, Ferritin, Fibrinogen, Procalcitonin,

It is anticipated that any or all of these markers will show significant differential regulation in inflammatory processes of the body tissues. A number of physiologically more important biomarkers of inflammatory processes were evaluated including: MMP-I, MMP-I2, MMP-I3, MMP-2, MMP-3, MMP-7, MMP-8, MMP-9, Collagen Type 1, Collagen Type 2, Collagen Type 4, Collagen Type 6, Glutathione S Transferase, HIF-1, NFKB, NFKB Gene Family, p53, PPAR, SMAD 2/3, Caspase 8 (active), Cytochrome c, EGF-Receptor, EGFR (Tyr), Erk 1/2 (Total), Rb (Total), PKC, PKB-a, Phospho PKC a, PDGF Receptor-a, PDGF Receptor-p, PDGF receptor-p (Tyr75I ), Fatty Acid Binding Protein, FGF-2, TNF-α, TNF-P, TNF-RI, TNFRII, TNFSF5, TRAIL, VCAM-I,VEGF, TG F alpha, Adiponectin, C-Peptide, C-Reactive Protein, Complement Factor D, CRF, FGF-9, FSH, Erk/MAPKK, Erk2 (total), Fibrinogen, CRP, IFN-gamma, IL-IO, IL-I beta, IL-6, IL-8, HSP-27 Total, IKB-a (phospho), IKB-a (Ser32/Ser36), IKB-a pS32, IKB-a Total, IKBa (serS32), MMP-9, serum Amyloid A, Serum Amyloid P, JNK Total, TNF-a, NG F, EGF, Insulin, IL-I2, IL-I2 p40, IL-I2 p40/p70, IL-I2 p70, IL-I3, IL-15, IL-16, IL-17, IL-18, IL-23, IL-25, IL-1 alpha, IL-1 beta, IL-Ira, IL-I ra/IL-I F3, IL-2, IL-2Ra, IL-3, IL-4, IL-5, IL-6, IL-7, IL-8, IL-9 and IL-10.

From these, 40 were identified as being most likely to be modulated under the irritancy conditions used in the experiment. These 40 markers were: MCP-1, VEGF, IL1ra, IL 12 p40, MCP 3, TNF α, MIP 1b, IL1α, G CSF, IL 6, IL 7, IL 2, Eotaxin, FGF basic, IL 10, IL 5, CRP, IFN y, IL 12 p70, ENA 78, IL 8, RANTES, IL 1p, MIP 1a, GMCSF, MIG, IL 4, IL 13, IP 1a, PDGF BB, GRO a, NGF, IL 17, TNF α, Prostaglandin E2, MMP 13, MMP 1, MMP 2, MMP 9, MMP 3.

It can be anticipated that significant numbers of additional primary and secondary inflammatory mediators will be identified among the foregoing sets of markers based on known up and downstream partners of these molecules in biomolecular signaling. From these 40, the following 32 were selected as most significantly modulated relative to controls: MCP-I, VEGF, IL1ra, IL 12 p40, MCP 3, IL1-α, IL1β, TNF α, MIP 1b, GCSF, IL 6, IL 7, IL 2, Eotaxin, FGF basic, IP 10, IL 4, CRP, IFN γ, IL 12 p70, ENA 78, IL 8, Prostaglandin E2, MMP 1, MMP 13, MMP 2, MMP 9 and MMP 3, RANTES, PDGF BB, GROa.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention.

Although the present invention has been described in terms of the foregoing embodiments, such description has been for exemplary purposes only and, as will be apparent to those of ordinary skill in the art, many alternatives, equivalents, and variations of varying degrees will fall within the scope of the present invention. That scope, accordingly, is not to be limited in any respect by the foregoing description; rather, it is defined only by the claims that follow.

We claim:

1. A dicarboxylic acid ester composition, comprising:
R₂OOC—(CH₂)n-COOR₁
wherein:
a) R₁ is selected from a group consisting of hydrogen, alkyl groups of up to about 18 carbon atoms, aryl groups of up to about 18 carbon atoms, alkylene group of up to about 18 carbon atoms and an arylene group of up to about 18 carbon atoms;
b) R₂ is selected from a group consisting of hydrogen, alkyl groups of up to about 18 carbon atoms, aryl groups of up to about 18 carbon atoms, alkylene group of up to about 18 carbon atoms and an arylene group of up to about 18 carbon atoms; and
c) n is between 5 and 16 carbon atoms.

2. The dicarboxylic acid ester composition according to claim 1 wherein deuterium may be substituted in any position for hydrogen.

3. The dicarboxylic acid ester composition according to claim 1 wherein the alkyl, aryl and alkylene groups of R₁ may be substituted or unsubstituted, branched or straight chains and R₁ may contain heteroatoms and may be straight chained or branched.

4. The dicarboxylic acid ester composition according to claim 1 wherein the alkyl, aryl and alkylene groups of R₂ may be substituted or unsubstituted, branched or straight chains and R₂ may contain heteroatoms and may be straight chained or branched.

5. The dicarboxylic acid ester composition according to claim 1 wherein R1 is selected from methyl, ethyl, 1 or 2 propyl, 1 or 2 butyl, t-butyl, 1, 2 or 3 pentyl, 1, 2, or 3 hexyl, 1, 2, 3, or 4 heptyl, 1, 2, 3 or 4 octyl, 1, 2, 3, 4 or 5 nonyl, 1, 2, 3, 4, or 5 decyl, 1, 2, 3, 4, 5, or 6 undecyl, cyclohexyl and ring substituted variations thereof, benzyl and ring substituted variations thereof, phenethyl and substituted variations thereof and R2 is hydrogen.

6. The dicarboxylic acid ester composition according to claim 1 wherein R1 and R2 are selected from methyl, ethyl, 1 or 2 propyl, 1 or 2 butyl, t-butyl, 1, 2 or 3 pentyl, 1, 2, or 3 hexyl, 1, 2, 3, or 4 heptyl, 1, 2, 3 or 4 octyl, 1, 2, 3, 4 or 5 nonyl, 1, 2, 3, 4, or 5 decyl, 1, 2, 3, 4, 5, or 6 undecyl, cyclohexyl and ring substituted variations thereof, benzyl and ring substituted variations thereof, phenethyl and substituted variations thereof and R1 is the same as R2.

7. A method of treating a disease having an inflammatory component, comprising:
formulating a treatment comprising a dicarboxylic acid ester composition in a pharmaceutically acceptable vehicle or diluent; and
administering the treatment to a patient suffering from an inflammatory condition of body tissues.

8. The method of treating a disease having an inflammatory component according to claim 7, further comprising:
re-administering the treatment to the patient until the body tissues substantially ceases to exhibit inflammation.

9. The method of treating a disease having an inflammatory component according to claim 7, wherein administering the treatment comprises delivering the treatment to a patient in need thereof by any of the following routes: intravenous, intraperitoneal, intrathecal, trans-placental, vaginal, rectal, transdermal, topical, nasal, oral, or parenteral.

10. The method of treating a disease having an inflammatory component according to claim 7, wherein the dicarboxylic acid ester composition comprises a preferred amount of a mixture of azelaic acid esters chosen specifically to complement and counteract a particular inflammatory condition being treated.

11. The method of treating a disease having an inflammatory component according to claim 7, wherein the percent concentration of azelaic acid esters is between 5% and 25% by weight.

12. The method of treating a disease having an inflammatory component according to claim 7, wherein the percent concentration of azelaic acid esters is between 10% and 20% by weight.

13. The method of treating a disease having an inflammatory component according to claim 7, wherein formulating the treatment further comprises combining the dicarboxylic acid ester composition in the pharmaceutically acceptable vehicle or diluent with at least one pharmacologically active compound.

14. The method of treating a disease having an inflammatory component according to claim 7 further comprising administering the treatment in combination with at least one pharmacologically active compound.

15. The method of treating a disease having an inflammatory component according to claim 7 wherein the dicarboxylic acid ester composition, comprises:
R₂OOC—(CH₂)n-COOR₁
wherein:
a) R₁ is selected from a group consisting of hydrogen, alkyl groups of up to about 18 carbon atoms, aryl groups of up to about 18 carbon atoms, alkylene group of up to about 18 carbon atoms and an arylene group of up to about 18 carbon atoms;
b) R₂ is selected from a group consisting of hydrogen, alkyl groups of up to about 18 carbon atoms, aryl groups of up to about 18 carbon atoms, alkylene group of up to about 18 carbon atoms and an arylene group of up to about 18 carbon atoms; and
c) n is between 5 and 16 carbon atoms.

16. The method of treating a disease having an inflammatory component according to claim 15 wherein deuterium may be substituted in any position for hydrogen.

17. The method of treating a disease having an inflammatory component according to claim 15 wherein the alkyl, aryl and alkylene groups of R₁ may be substituted or unsubstituted, branched or straight chains and R₁ may contain heteroatoms and may be straight chained or branched.

18. The method of treating a disease having an inflammatory component according to claim IS wherein the alkyl, aryl and alkylene groups of R₂ may be substituted or unsubstituted, branched or straight chains and R₂ may contain heteroatoms and may be straight chained or branched.

19. The method of treating a disease having an inflammatory component according to claim 15 wherein R1 is selected from methyl, ethyl, 1 or 2 propyl, 1 or 2 butyl, t-butyl, 1, 2 or 3 pentyl, 1, 2, or 3 hexyl, 1, 2, 3, or 4 heptyl, 1, 2, 3 or 4 octyl, 1, 2, 3, 4 or 5 nonyl, 1, 2, 3, 4, or 5 decyl, 1, 2, 3, 4, 5, or 6 undecyl, cyclohexyl and ring substituted variations thereof, benzyl and ring substituted variations thereof, phenethyl and substituted variations thereof and R2 is hydrogen.

20. The method of treating a disease having an inflammatory component according to claim 15 wherein R1 and R2 are selected from methyl, ethyl, 1 or 2 propyl, 1 or 2 butyl, t-butyl, 1, 2 or 3 pentyl, 1, 2, or 3 hexyl, 1, 2, 3, or 4 heptyl, 1, 2, 3 or 4 octyl, 1, 2, 3, 4 or 5 nonyl, 1, 2, 3, 4, or 5 decyl, 1, 2, 3, 4, 5, or 6 undecyl, cyclohexyl and ring substituted variations thereof, benzyl and ring substituted variations thereof, phenethyl and substituted variations thereof and R1 is the same as R2.

21. The method of treating a disease having an inflammatory component according to claim 7, further comprising:
obtaining a sample of tissue or body fluid from the patient suffering from inflammation;
measuring the concentration of one or more a biomarkers for the tissue or body fluid from the patient suffering from inflammation;
comparing the concentration of the one or more biomarkers for the tissue or body fluid from the patient suffering from inflammation to one or more biomarkers for tissue or body fluid from a patient not suffering from inflammation;
employing the comparison of inflamed tissue to normal tissue biomarker concentrations to determine and guide the selection of a particular azelaic acid ester composition and other pharmacologically active compounds as appropriate for the treatment of the disease manifesting the particular inflammatory biomarker pattern;
formulating a new dicarboxylic acid ester composition, with or without the addition of additional pharmacologically active compounds, based on the observed effects and effectiveness of the administered dicarboxylic acid ester composition as illustrated by the biomarker comparison; and
administering the new dicarboxylic acid ester composition, with or without additional pharmacologically active compounds, to the patient suffering from inflammation.
